(19) United States
(12) Patent Application Publication
(10) Pub. No.: US 2026/0356883 A1
(43) Pub. Date: Nov. 12, 2026

(54) OPTIMIZATION OF ELUTION CONDITIONS

(71) Applicant: Cytiva BioProcess R&D AB, Uppsala (SE)

(72) Inventors: Tomas Bjorkman, Uppsala (SE); Gustav Rodrigo, Uppsala (SE); Ulrika Knutsson, Uppsala (SE)

(21) Appl. No.: 19/153,583

(22) PCT Filed: Jan. 30, 2024
(86) PCT No.: PCT/EP2024/052206

§ 371 Date: Aug. 4, 2025

(30) Foreign Application Priority Data
Feb. 6, 2023 (SE) 2350112-5

(51) Int. Cl. C07K 1/22

(52) U.S. Cl. CPC C07K 1/22

(57) ABSTRACT

The present invention relates to a method of purifying an antibody or an antibody fragment, adsorbing at least one of the antibody or the antibody fragment onto an affinity separation matrix by contacting a liquid sample with the affinity separation matrix; wherein the affinity matrix is an affinity separation matrix comprises a ligand based on Protein L, or any variation thereof that binds to a κ-light chain of the antibody or antibody fragment, and separating the at least one antibody or antibody fragment from the affinity separation matrix by using an elution buffer, wherein the elution buffer has concentration of 5-50 mM. It equally relates to a method for separation of bispecific antibodies.

### TECHNICAL FIELD

The present invention relates to the field of chromatography, more particularly to affinity chromatography. More specifically, it relates to elution conditions for Protein L separation matrices and how to improve said conditions.

### BACKGROUND

Immunoglobulins represent the most prevalent biopharmaceutical products in either manufacture or development worldwide. The high commercial demand for and hence value of this particular therapeutic market has led to the emphasis being placed on pharmaceutical companies to maximize the productivity of their respective mAb manufacturing processes whilst controlling the associated costs.

Affinity chromatography is used in most cases as one of the key steps in the purification of these immunoglobulin molecules, such as monoclonal or polyclonal antibodies. A particularly interesting class of affinity reagents is proteins capable of specific binding to invariable parts of an immunoglobulin molecule, such interaction being independent on the antigen-binding specificity of the antibody. Such reagents can be widely used for affinity chromatography recovery of immunoglobulins from different samples such as, but not limited to, serum or plasma preparations, or cell culture derived feedstocks.

For immunoglobulins, immunoglobulin fragments, antibodies, or antibody fragments, such as Fab, single-chain variable fragments (scFv), bi-specific T-cell engagers (BiTEs), domain antibodies etc., which lack the Fc chain but have a subclass 1,3 or 4 kappa light chain, matrices comprising Protein L derived from Finegoldia magna (formerly Peptostreptococcus Magnus) (B Åkerström, L Björck: J. Biol. Chem. 264, 19740-19746, 1989; W Kastem et al: J. Biol. Chem. 267, 12820-12825, 1992; B H K Nilson et al: J. Biol. Chem. 267, 2234-2239, 1992 and U.S. Pat. No. 6,822,075) show great promise as a purification platform providing the high selectivity needed.

Protein L matrices are commercially available as for instance Capto™ L and MabSelect™ VL from Cytiva™ and can be used for separation of kappa light chain-containing proteins such as intact antibodies, Fabs, scFv fragments, domain antibodies etc. About 75% of the antibodies produced by healthy humans have a kappa light chain and about 90% of therapeutic monoclonal antibodies and antibody fragments contain kappa light chains (Carter, P., Lazar, G. Next generation antibody drugs: pursuit of the ‘high-hanging fruit’. Nat Rev Drug Discov 17, 197-223 (2018). https://doi.org/10.1038/nrd.2017.227).

In most commercially available processes, the target will bind to the ligand of the affinity matrix such that normally a pH of 2.5-3.5 is necessary in order for the target to dissociate from the affinity ligand and be eluted with the elution buffer. Such low pH risks compromising the target protein, such as causing aggregation of the target protein, denaturation of the target protein, etc. These issues will lead to a lower productivity and yield of correct and uncompromised target protein. Thus, regardless of the target to be purified or separated by a chromatographic purification or separation process, it is of high interest to be able to elute the target at a pH closer to neutral pH than previously, thereby minimizing the risks discussed above. U.S. Pat. No. 10,844,112 B2 discusses the use of a specific buffer for elution. Previous attempts have also been made by adding salt to the elution buffer to increase the elution pH, however such an approach requires removal of the high levels of salt from the eluate. Thus, there is still of great interest to find alternative ways of eluting target molecules at higher pH than previously.

### DEFINITIONS

The terms “antibody” and “immunoglobulin” may be used interchangeably herein and refers to an antigen-binding protein having a basic four-polypeptide chain structure consisting of two heavy (H) chains and two light (L) chains, said chains being stabilized by interchain or intrachain disulfide bonds. Each heavy chain is comprised of a heavy chain variable region (abbreviated herein as HCVR or VH) and a heavy chain constant region (CH). The heavy chain constant region is comprised of three domains, CH1, CH2 and CH3. Each light chain is comprised of a light chain variable region (VL) and a light chain constant region. The light chain constant region is comprised of one domain, CL. There are two types of light chain in humans, kappa chain and lambda chain. The term is to be understood to include any antibody, including but not limited to monoclonal antibodies, bi-specific antibodies, multi-specific antibodies, as well as fragments of antibodies, fusion proteins comprising antibodies or antibody fragments and conjugates comprising antibodies or antibody fragments, such as Antibody-Drug Conjugates (ADC).

The term “mAb” stands for monoclonal antibody.

The term “Fc region” refers to a C-terminal region of an IgG antibody, particularly the C-terminal region of the heavy chain(s) of said IgG antibody. The term “Fc binding” refers to the capability to bind to said region.

The term “kappa light chain-containing protein” is used as a synonym of “immunoglobulin kappa light chain-containing protein” and herein means a protein comprising a subclass 1, 3 or 4 kappa light chain (also called V_{κI}, V_{κIII} and V_{κIV}, as in B H K Nilson et al: J. Biol. Chem. 267, 10 2234-2239, 1992) derived from an antibody and includes any intact antibodies, antibody fragments, fusion proteins, conjugates or recombinant proteins containing a subclass 1, 3 or 4 kappa light chain.

The term “Fab” or “Fab fragment” refers to the antigen-binding region and includes both a constant domain and the variable domains of both the heavy and light chains. A Fab may comprise a kappa light chain and/or a lambda light chain.

The term “Fv fragment” refers to the fragment variable region and contains only the two variable domains, VH and VL. The VH and VL are held together in Fv fragments by non-covalent interactions.

The term “bi-specific antibody” stands for an antibody that can bind to two different types of antigen or two different epitopes on the same antigen. Likewise, a tri-specific antibody stands for an antibody that can bind to three different types of antigen or three different epitopes on the same antigen. The term “multi-specific antibody” stands for an antibody that can bind more than two different types of antigen or more than two different epitopes on the same antigen. A bi-specific or multi-specific antibody is a heterodimer, with differing variable regions accounting for the bi- or multi-specificity, as opposed to a mAb which is a homodimer.

The terms “Fc-binding polypeptide”, “Fc-binding agent” and “Fc-binding protein” mean a polypeptide, molecule or protein, respectively, capable of binding to the crystallizable part, the Fc-region, of an antibody and includes, but is not limited to, e.g. Protein A and Protein G, or any fragment or fusion protein thereof that has maintained said binding property.

The term “VH binding” refers to the capability to bind to the VH region of an antibody or an antibody fragment.

The term “liquid sample” as used herein, refers to a liquid containing at least one target substance which is sought to be purified from other substances also present. Liquid samples can, for example, be aqueous solutions, organic solvent systems, or aqueous/organic solvent mixtures or solutions. The source liquids are often complex mixtures or solutions containing many biological molecules (such as proteins, antibodies, hormones, and viruses), small molecules (such as salts, sugars, lipids, etc.) and even particulate matter. While a typical source liquid of biological origin may begin as an aqueous solution or suspension, it may also contain organic solvents used in earlier separation steps such as solvent precipitations, extractions, and the like. Examples of liquid samples that may contain valuable biological substances amenable to the purification by various embodiments of the present invention include, but are not limited to, a culture supernatant from a bioreactor, a homogenized cell suspension, plasma, plasma fractions, and milk. Alternatively, the liquid sample may be referred to as “feed”, “Clarified Cell Culture Feed” or “CCF”.

A “buffer” is a substance which, by its presence in solution, increases the amount of acid or alkali that must be added to cause unit change in pH. A buffered solution resists changes in pH by the action of its acid-base conjugate components. The term “physiological pH” refers to the pH of mammalian blood (i.e., 7.38 or about 7.4). Thus, a physiologic pH range is from about 7.2 to 7.6. Traditional buffer components include, but are not limited to, organic and inorganic salts, acids and bases. Exemplary buffers for use in purification of biological molecules (e.g., protein molecules) include the zwitterionic or “Good” Buffers, see e.g., Good et al. (1966) Biochemistry 5:467 and Good and Izawa (1972) Methods Enzymol. 24:62. Commonly used acidic buffers in antibody processes are based on carboxylic acids.

“Washing liquid” or “wash buffer” refer herein to the liquid used to carry away impurities from the chromatography resin to which is bound the target substance. More than one wash liquid can be employed sequentially, e.g., with the successive wash liquids having varying properties such as pH, conductivity, solvent concentration, etc., designed to dissociate and remove varying types of impurities that are non-specifically associated with the chromatography resin.

“Binding buffer” refers to a buffer solution intended for loading of the target molecule onto the chromatography column.

The term “equilibration buffer” refers in the present disclosure to a buffer used to prepare the affinity matrix, with bound target protein, for the elution. Equilibration buffer may also be used for washing the affinity matrix with bound target protein.

“Elution liquid” or “elution buffer”, which are used interchangeably herein, refers herein to the liquid that is used to dissociate the target substance from the chromatography resin, thereby eluting the binding region-containing protein from the immobilized binding agent, after it has been washed with one or more wash liquids. The elution liquid acts to dissociate the target substance without denaturing it irreversibly. Typical elution liquids are well known in the chromatography art and may have a different pH (typically lower pH), higher concentrations of salts, free affinity ligands or analogs, or other substances that promote dissociation of the target substance from the chromatography resin. “Elution conditions” refers to process conditions imposed on the target substance-bound chromatography resin that dissociate the target substance from the chromatography resin, such as the contacting of the target substance-bound chromatography resin with an elution liquid or elution buffer to produce such dissociation.

Preferably the elution buffer has a low pH and thereby disrupts interactions between separation matrix and the protein of interest. Typically, the low pH elution buffer has a pH in the range from about 2 to about 5, such as in the range from about 3 to about 4. Examples of buffers that will control the pH within this range include glycine, phosphate, acetate, and citrate buffers, as well as combinations of these. Commonly used buffers are citrate and acetate buffers, most preferably sodium citrate or sodium acetate buffers.

Ionic strength is calculated according to the following formula well-known within the technical field:

$$I = \frac{1}{2} \sum ( {c}_{i} \cdot {z}_{i}^{2} )$$

$$where :$$

$$I = Ionic strength ;$$

$$\sum = Sum of values ;$$

$${c}_{i} = Concentration of ions ; and$$

$${z}_{i}^{2} = Charges of ions squared .$$

As used herein, the terms “comprises”, “comprising”, “containing”, “having” and the like can mean “includes”, “including”, and the like; “consisting essentially of” or “consists essentially” is an open-ended term, allowing for the presence of more than that which is recited so long as basic or novel characteristics of that which is recited is not changed by the presence of more than that which is recited, but excludes prior art embodiments.

In the absence of specific temperature data indicating otherwise, all measurements and methods are performed at room temperature (22+/−2 °C).

### SUMMARY OF THE INVENTION

It has been an objective for the present inventors to find a process wherein the elution conditions allow for a milder pH, i.e. higher than normally used, or less acidic pH, at the elution step for a target molecule from an affinity matrix.

Additionally, it has been an objective to design a process whereby the target compound is well separated from other molecules in a feed. Such a separation is particularly interesting for separating bi-specific heterodimeric antibodies of interest from other antibodies that do not have a desired configuration of heavy chains and light chains, e.g. homodimers.

The present inventors have surprisingly found that using elution buffers at a lower concentration than usual, and thus with a lower ionic strength, leads to a less acidic elution pH of the target molecule in a purification process of antibodies.

Thus, according to a first aspect, herein is provided a method for purification of an antibody or an antibody fragment, comprising the steps of:
adsorbing at least one of the antibody or the antibody fragment onto an affinity separation matrix by contacting a liquid sample with the affinity separation matrix;
washing the affinity separation matrix to remove impurities;
equilibrating the affinity separation matrix with an equilibration buffer
separating the at least one antibody or antibody fragment from the affinity separation matrix by using an elution buffer,
wherein the affinity separation matrix comprises a ligand based on Protein L, or any variation thereof that binds to a κ-light chain of the antibody or antibody fragment, and wherein the elution buffer has concentration of 5-50 mM.

According to a second aspect, herein is provided a method for separation of bispecific antibodies comprising the steps of:
adsorbing a feed comprising at least the bispecific antibody onto an affinity separation matrix by contacting a liquid sample with the affinity separation matrix;
washing the affinity separation matrix to remove impurities;
equilibrating the affinity separation matrix with an equilibration buffer
adding an elution buffer to the separation matrix to elute the bispecific antibody from the affinity separation matrix,
wherein the affinity separation matrix comprises a ligand based on Protein L, or any variation thereof that binds to a κ-light chain of the bispecific antibody, and wherein the elution buffer has a concentration of 5-50 mM.

According to a third aspect, herein is provided a method for obtaining an elution pH of above 4 from a Protein L or Protein L-derived chromatography separation matrix using an elution buffer based on a carboxylic acid, wherein the elution pH is obtained by lowering the elution buffer concentration to below 50 mM, preferably to between 10-50 mM.

Relating to all aspects, the elution buffer may comprise a buffering dicarboxylic acid. The elution buffer may be a succinate buffer solution. The succinate buffer solution may have a concentration of 10-50 mM. The pH of the succinate buffer solution may be 5-3, or 5-2.8.

Alternatively, the elution buffer may comprise a buffering tricarboxylic acid. The elution buffer may be a citrate buffer solution. The citrate buffer solution may have a concentration of 5-45 mM. The pH of the citrate buffer solution may be 4.5-2.5.

Alternatively, the elution buffer may comprise a buffering monocarboxylic acid. The elution buffer may be a propionate buffer solution.

Relating to all aspects, the elution is preferably performed in a pH range of from about 5 to about 3.

### DRAWINGS

FIG. 1 is an overlay of chromatograms run on protein L columns with Trastuzumab, with the indicated citrate elution buffers. The chromatograms are zoomed in on the elution peaks.

FIG. 2 is an overlay of chromatograms run on Protein L columns with Trastuzumab, with 50 mM succinate elution buffer and the indicated concentrations for the equilibration buffers. The chromatograms are zoomed in on the elution peaks.

FIG. 3 is an overlay of chromatograms for a bispecific antibody (bsAB, as specified in the Examples) on a Protein L separation matrix comparing the use of a 10 mM citrate elution buffer and a 10 mM equilibration/50 mM elution succinate buffer. The chromatograms are zoomed in on the elution peaks.

FIG. 4 relates to the SEC analysis of eluted fractions from FIG. 1 and FIG. 2. FIG. 4A indicates where the fractions were collected for the 10 mM/50 mM Succinate example. FIG. 4B shows the SEC results of the fractions indicated in FIG. 4A.

FIG. 5 shows chromatograms for a bispecific antibody on a Protein L separation matrix using a 15 mM succinate elution buffer. The right-hand scale is pH. FIG. 5A shows the elution profile with a pH gradient, and FIG. 5B shows the elution profile with a step-wise elution.

FIG. 6 shows chromatograms for a bispecific antibody on a Protein L separation matrix using a 50 mM propionate elution buffer. The right-hand scale is pH. FIG. 6A shows the elution profile with a pH gradient, and FIG. 6B shows the elution profile with a step-wise elution.

FIG. 7 shows chromatograms run on Protein L columns with Trastuzumab. The chromatograms are zoomed in on the elution peaks. The right-hand scale is pH. FIG. 7A shows a stepwise elution using a 15 mM Succinate elution buffer. FIG. 7B shows a stepwise elution using a 50 mM propionate elution buffer.

FIG. 7C shows a stepwise elution using a 20 mM Citrate elution buffer.

### DETAILED DESCRIPTION

The present inventors have had as an objective to find a process wherein the elution conditions allow for a milder pH, i.e. higher than normally used, at the elution step for a target molecule from an affinity matrix.

The inventors have surprisingly found that low concentrations of an elution buffer are advantageous in obtaining a milder elution pH, a less acidic elution pH, for a target molecule compared to higher concentrations of the same buffer in a purification process of antibodies.

Thus, the present invention relates in a first aspect to a method of purifying an antibody or an antibody fragment, comprising the steps of:
adsorbing at least one of the antibody or the antibody fragment onto an affinity separation matrix by contacting a liquid sample with the affinity separation matrix;
washing the affinity separation matrix to remove impurities;
equilibrating the affinity separation matrix with an equilibration buffer
separating the at least one antibody or antibody fragment from the affinity separation matrix by using an elution buffer,
wherein the elution buffer has concentration of 5-50 mM.

The affinity separation matrix comprises a ligand based on Protein L, or any variation thereof that binds to a κ-light chain of the antibody or antibody fragment. In an alternative language, the separation matrix is a Protein L or Protein L-derived separation matrix. In the foregoing, the separation matrix will, for convenience, be referred to as a Protein L separation matrix.

Any buffer solution used for the equilibration buffer and the elution buffer in the methods disclosed herein must be of a substance that works well as a buffer That is that the substance has good buffering characteristics (see under “Definitions” above). The skilled person will be aware of substances that have good buffering characteristics, and which do not, and which of such substances that are applicable to the present method.

The elution buffer may preferably be a monocarboxylic acid, a dicarboxylic acid, or a tricarboxylic acid. Dicarboxylic acids in a solution will lead to two carboxyl groups per acid molecule being present in a solution. Tricarboxylic acids in a solution will lead to three carboxyl groups per acid molecule being present in a solution. Thus, the more carboxylic groups present, the more counterions will be needed to achieve the desired pH, thereby leading to a higher ionic strength.

The monocarboxylic acid used in the method disclosed herein may be e.g. formic acid, acetic acid and propionic acid, and preferably formic acid or propionic acid. Preferably, propionic acid is used as the elution buffer. An elution buffer may thus preferably be a propionate solution of 50 mM or lower. However, for the propionate buffer, also higher concentrations, such as 75 mM or 100 mM will also lead to a higher elution pH, as compared to the commonly used citrate buffer.

The dicarboxylic acid used in the method may be e.g. oxalic acid, malonic acid, and succinic acid. Preferably succinic acid is used as the elution buffer. An elution buffer may thus preferably be a succinate solution of 10-50 mM, such as 10 mM, or 15 mM, or 20 mM, or 25 mM, or 30 mM, or 35 mM, or 40 mM, or 45 mM, or 50 mM.

The elution of the target molecule is typically performed by decreasing the pH. This may be performed with a decreasing pH gradient or in a step-wise manner. The succinate elution buffer may have a pH of 5-3, or 5-2.8. By gradually increasing the amount of elution buffer in relation to an equilibration buffer, a decreasing pH is achieved, and elution will occur. The equilibration buffer should preferably have a pH of above 5, such as 5.5, or about 5.8-6. The elution occurs within a pH range of from about 5 to about 3.

In the examples herein, an equilibration buffer comprising the same buffer salt or acid as the elution buffer is used. This is to ensure a linear gradient in the examples and to better visualize the advantage of the elution conditions. However, the skilled person will realize that any commonly used equilibration buffer may be used, such as Phosphate buffered saline (PBS) with varying concentrations of NaCl, Tris buffered saline etc. For the current invention, it is the elution buffer that is important in affecting the increased elution pH of the target molecule. It should be noted however, that the present invention focuses on the buffer solution for the desired impact on elution pH. Additional salt is not used to increase or impact the elution pH according to the methods disclosed herein. Hence, the increase of elution pH is according to the present disclosure achieved without additional salt.

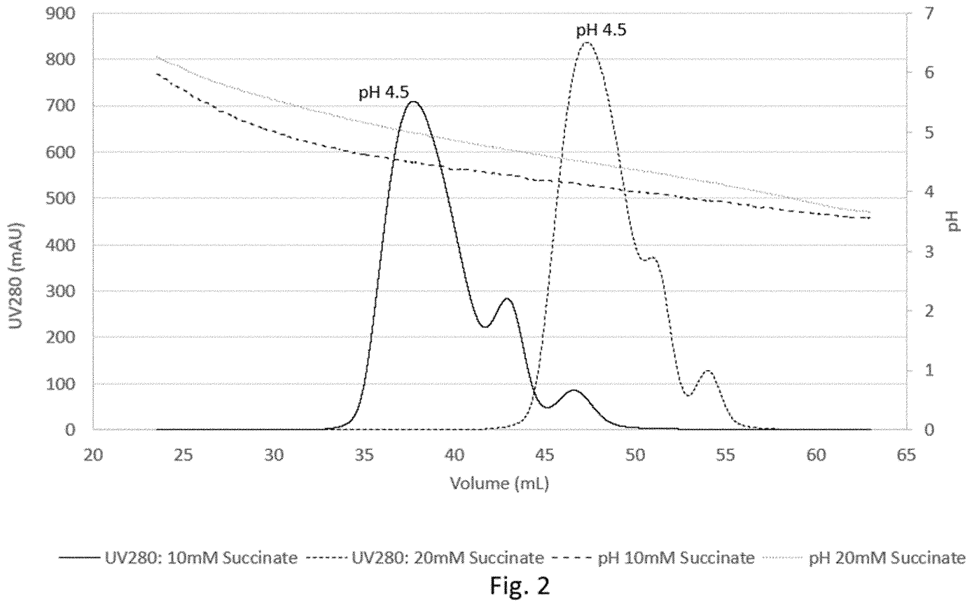

As can be seen in FIG. 2, elution with a succinate solution as disclosed above leads to an elution pH of around 4.5 for a Protein L separation matrix. This is significantly higher than what has previously been achieved with for instance acetate buffers, or citrate buffers with a concentration at or above 50 mM. For instance, when using citrate at concentrations at or higher than 50 mM, it has previously been observed that the elution pH decreases, which is undesirable.

The tricarboxylic acid used in the method may preferably be a citrate buffer solution. The elution buffer may be a citrate solution of 5-45 mM, such as 5 mM, or 10 mM, or 15 mM, or 20 mM, or 25 mM, or 30 mM, or 35 mM, or 40 mM, or 45 mM.

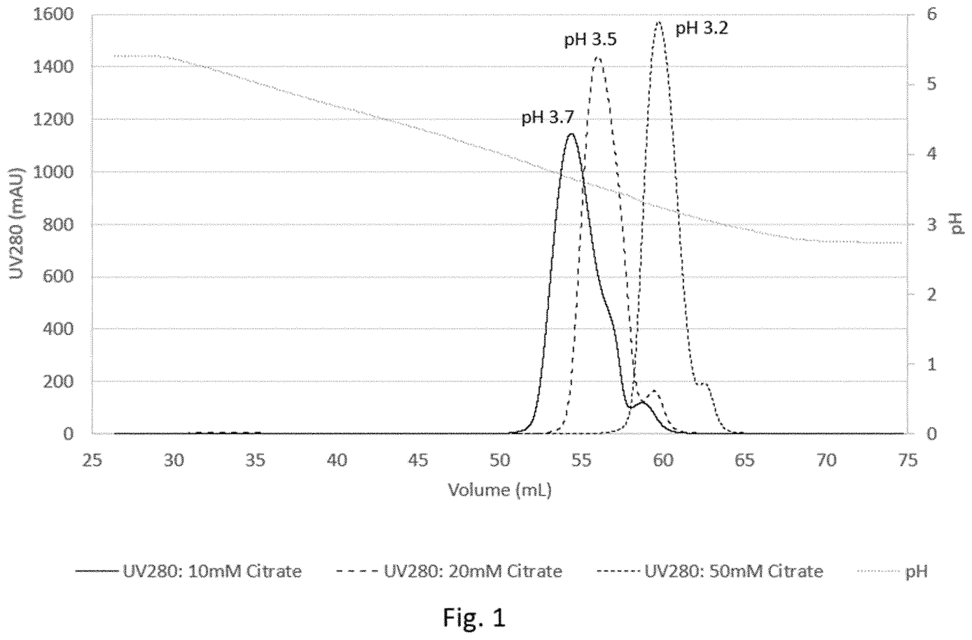

The citrate elution buffer may have a pH of 4.5-2.5. By gradually increasing the amount of elution buffer in relation to the equilibration buffer, a decreasing pH is achieved, and elution will occur. The equilibration buffer should have a pH of about or above 5, such as 5.5, or 6.0. The elution occurs within a pH range of from about 5 to about 3. As can be seen in FIG. 1, an elution pH of up to 3.7 can be achieved with the citrate buffer solutions having the lowest concentrations when using a Protein L separation matrix. This is in contrast to the disclosure of U.S. Pat. No. 10,844,112 B2, wherein a citrate buffer is disclosed as less advantageous for a Protein L affinity matrix all together, and an elution pH of below 3.0 is shown for a 100 mM citrate elution buffer. By using a lower concentration of the citrate buffer according to the present disclosure, a higher and more advantageous elution pH is thus achieved, as shown in FIG. 1. Additionally, the lowest concentration of citrate buffer shown herein leads to a higher elution pH than the shown highest elution pH in U.S. Pat. No. 10,844,112 B2 using an acetate buffer (pH 3.6).

Thus, the advantage of using a low concentration for the elution buffer according to the present invention and thereby achieving a low ionic strength, is that the pH at which elution occurs may thereby be elevated, as can be seen in the Examples herein.

In a second aspect, the invention relates to a method for separation of bispecific antibodies comprising the steps of:
adsorbing a feed comprising at least the bispecific antibody onto an affinity separation matrix by contacting a liquid sample with the affinity separation matrix;
washing the affinity separation matrix to remove impurities;
equilibrating the affinity separation matrix with an equilibration buffer
adding an elution buffer to the separation matrix to elute the bispecific antibody from the affinity separation matrix,
wherein the elution buffer has concentration of 5-50 mM.

The elution buffer in this second aspect is as disclosed in connection with the first aspect above.

The pH range for elution is as disclosed in connection with the first aspect.

The separation matrix in this second aspect is as disclosed in connection with the first aspect above.

The elution buffer is added in a pH gradient from about pH 6-5.8 to about pH 2.5-2.8. For instance, when using a succinate buffer, the pH of a succinate equilibration buffer is preferably about 5.8-6.0, and the pH of the succinate elution buffer is from about 5 to about 3, or from about 5 to about 2.8. For instance, when using a citrate buffer, the pH of a citrate equilibration buffer is preferably about 5.5 and the pH of the citrate elution buffer is from about 4.5 to about 2.5, or from about 4.5 to about 2.8. For instance, when using a propionate buffer, the pH of a propionate equilibration buffer is preferably about 5.8-6.0, and the pH of a propionate elution buffer is from about 5 to about 3, or from about 5 to about 3.5.

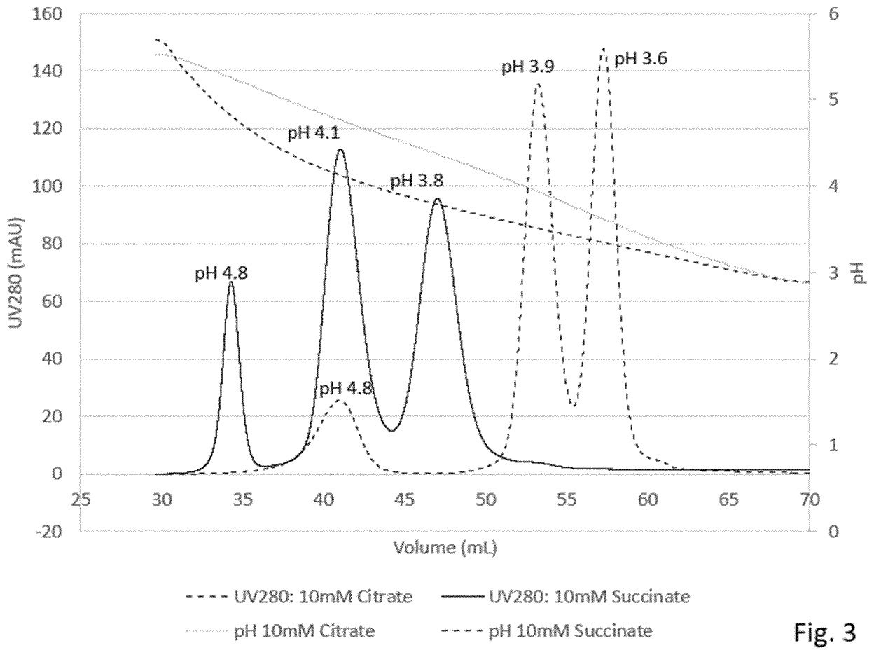

As can be seen in FIG. 3, the elution conditions using both the succinate buffer and the citrate buffer offers a clear separation of the homo- and heterodimers of the bispecific antibody on a Protein L separation matrix. Peak 1, at the highest elution pH, may correspond to undesired species that bind very lightly to the separation matrix, such as unspecific binding. Peak 2, at the second highest elution pH, corresponds to the heterodimeric bsAb having 1 kappa VL chain and 1 lambda VL chain, which is the bsAb that is the target for purification and separation. This has been confirmed by LC-MS data (not shown). Peak 3 corresponds to a homodimeric species having 2 kappa VL chains, also having been confirmed by LC-MS data (not shown). As was the case with the first aspect, the elution pH when using the succinate buffer is higher than when using the citrate buffer. However, both types of buffers enables an elution pH of 3.6 or above, or even around 4 for succinate, and consequently both offer a good separation at an advantageous pH.

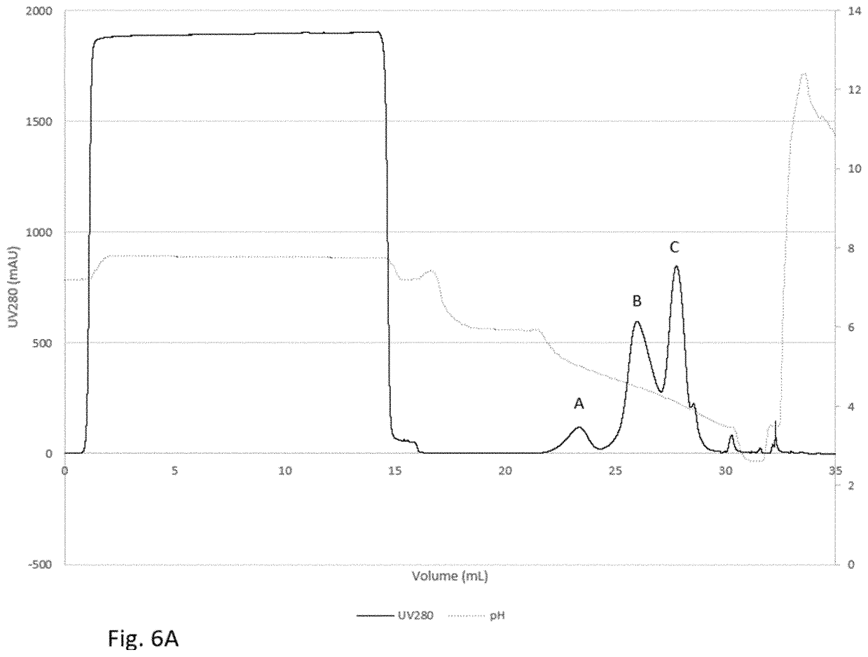

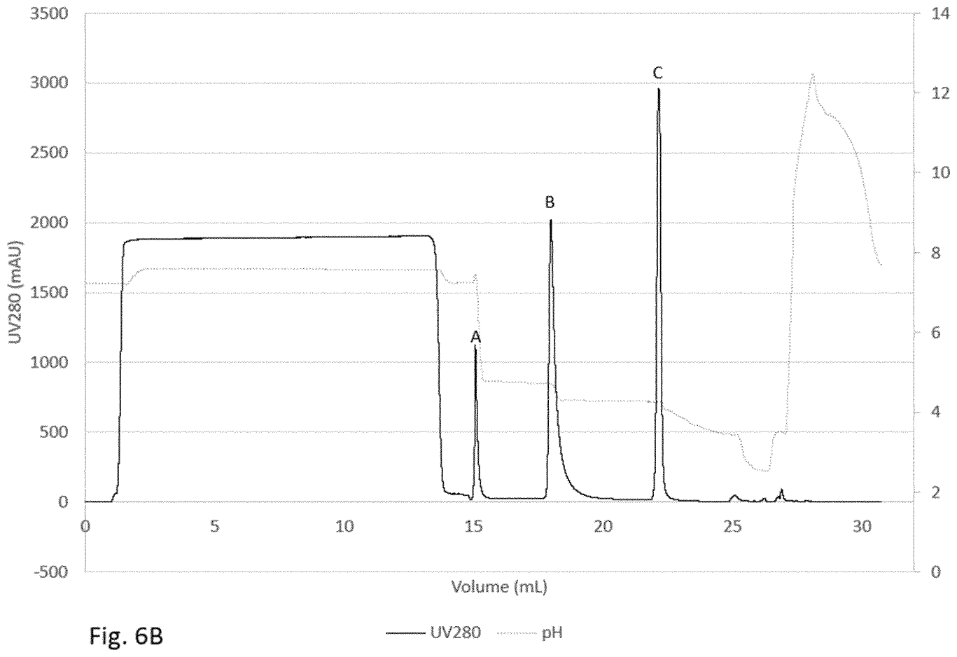

Additionally, FIGS. 6A and 6B clearly shows that using a propionate buffer offers a clear separation of the homo- and heterodimers of the bispecific antibody on a Protein L separation matrix, similarly to that described above. The separation is particularly clear for the stepwise elution in FIG. 6B.

According to a third aspect, herein is provided a method for obtaining an elution pH of above 4 from a Protein L or Protein L-derived chromatography separation matrix using an elution buffer based on a carboxylic acid, wherein the elution pH is obtained by lowering the elution buffer concentration to below 50 mM, preferably to between 10-50 mM. The composition of the elution buffer, as well as the pH and more specific concentrations for the elution buffer, is as disclosed in relation to the previous two aspects.

Thus, the methods of the present disclosure allows for elution of an antibody or antibody fragment from Protein L separation matrices at higher pH compared to what has been commonly used. This will be advantageous for the antibodies or antibody fragments as the elution conditions are closer to a neutral pH than previously shown methods, leading to lower risk of denaturation of the antibody or antibody fragment. Additionally, acidic pH might contribute to aggregation, which is mitigated by the methods of the present disclosure.

The present invention is further disclosed in the examples below. These examples should not be construed as to limit the scope of the invention to the exact settings or conditions shown therein but are merely included to illustrate the advantages of the present invention. The scope of the invention is defined by the appended claims, and any embodiment falling under those definitions, even if not explicitly disclosed, forms part of the invention.

### Examples

### Material

Sodium dihydrogen phosphate and disodium phosphate, Sodium chloride, Sodium hydroxide, Acetic acid, Citric acid, trisodium citrate, Succinic acid, disodium succinate hexahydrate, DL-Dithiothreitol (DTT), Tris(hydroxymethyl)aminomethane HCl and Tris(hydroxymethyl)aminomethane were purchased from Merck (Darmstadt, Germany). Formic acid 98-100% EMSURE® from Merck and Acetonitrile LC/MS GRADE (OPTIMA®) was purchased from Fisher Scientific. Propionic acid was purchased from Fluka Chemical, and Na-Propionate was purchased from Sigma-Aldrich.

### Equipment

An ÄKTA Pure 25 system controlled by Unicorn 7.7 (Cytiva™, Uppsala, Sweden) was used for all chromatographic runs. An ÄKTA Explorer 10 XT (Cytiva, Uppsala, Sweden) was used for SEC analysis. LC-MS instrument BioAccord (Waters), Acquity I-Class UPLC System coupled to Acquity RDa detector.

### Methods

### Protein L Chromatography

MabSelect VL (Protein L affinity resin, Cytiva™, Uppsala, Sweden) was packed in a 0.5 cm diameter column with 10 cm bed height. The column volume (CV) is approximately 2 mL. Detailed information for MabSelect VL chromatography is summarized in Table 1. Column load was either purified mAb, a clarified cell culture harvest of mAb, or a clarified cell culture harvest of bispecific Ab (bsAb). The mAb used in the experiments is Trastuzumab. The bsAb used is a bsAb comprising a kappa class 1 light chain from Trastuzumab, a lambda class 2 light chain from Avelumab, and Trastuzumab heavy chains, said bsAb being available from ThermoFisher. For runs with mAb purification, all runs were loaded at 10 mg of mAb per mL of resin and for runs with bsAb purification, all runs were loaded at 2.5 mg of bsAb per mL of resin. The column was loaded and ran in bind-elute mode. The target protein was eluted with elution buffer in a gradient from 0 to 100% over 20 CV. For all chromatographic runs, the system was run at a flow rate corresponding to a residence time of 6 min. All chromatograms were recorded by monitoring UV absorbance at 280 nm. Elution from selected runs was collected in fractions and analyzed by SEC for monomer purity and/or identity with LC-MS.

TABLE 1
MabSelect VL chromatography

| | Volume | |
|---|---|---|
| Step | (CV) | Buffer composition |
| Binding Buffer | 3 | 20 mM phosphate 150 mM NaCl pH 7.2 |
| Sample application | | mAb or bsAb as specified above |
| Salt wash | 7 | 20 mM phosphate 500 mM NaCl pH 7.2 |
| Wash/Equilibration | 3-5 | Buffer A. See Table 2 |
| Elution* | 5 | 0-100% Buffer B in 20 CV. See Table 2 |
| Strip | 3 | 0.5M acetic acid |
| Equilibration | 2 | 20 mM phosphate, 150 mM NaCl pH 7.2 |
| CIP (Clean In Place) | 3 | 0.1M NaOH |
| Re-equilibration | 1.5 | 20 mM phosphate 500 mM NaCl pH 7.2 |
| Re-equilibration | 3 | 20 mM phosphate 150 mM NaCl pH 7.2 |

TABLE 2
Equilibration buffers (Buffer A) and Elution Buffers (Buffer B)

| Experimental | Buffer A: | Buffer B: |
|---|---|---|
| setup | Equilibration buffer | Elution Buffer |
| 1 | 10 mM Succinate, pH 5.8 | 50 mM Succinate, pH 2.8 |
| 2 | 20 mM Succinate, pH 6 | 50 mM Succinate, pH 2.8 |
| 3 | 50 mM Citrate, pH 5.5 | 50 mM Citrate, pH 2.5 |
| 4 | 20 mM Citrate, pH 5.5 | 20 mM Citrate, pH 2.5 |
| 5 | 10 mM Citrate, pH 5.5 | 10 mM Citrate, pH 2.8 |
| 6 | 50 mM Propionate, pH 6 | 50 mM Propionate, pH 3.2 |
| 7 | 15 mM Succinate, pH 6 | 15 mM Succinate, pH 3.4 |

### Size-Exclusion Chromatography

SEC analysis was performed using a Superdex 200 Increase column (10×300 mm, Cytiva™). 50 μl of sample was injected per run. The mobile phase consisted of 200 mM Phosphate at pH 6.8. Elution of sample was performed isocratically for 30 min at a flow rate of 0.8 mL/min. UV absorbance at 280 nm was recorded for all chromatograms.

### Liquid Chromatography—Mass Spectroscopy (Data not Shown)

LC-MS analysis was performed using a BioResolve RP mAb Polyphenyl column (450 Å, 2.7 μm, 2.1×50 mm, Waters). Samples, diluted to 0.1 g/L, were reduced with DTT and digested with FabRICATOR® (Genovis) at 37 °C for 3 hours prior injection. 1 μl of sample was injected per run. The mobile phase consisted of 0.1% formic acid and the mAb or bsAb was eluted in a gradient using a mobile phase with 0.1% formic acid in acetonitrile at a flow of 0.5 mL/min for a total of 15 minutes. The column was kept at 60 °C during the analysis.

### Results

### Example 1-Purification of mAb Using Citrate Buffer and Protein L Separation Matrix

In this example, Protein L chromatography according to the above was performed with the experimental setups 3, 4 and 5 in Table 2. The resulting chromatogram is shown in FIG. 1. It is clear that the lower the concentration of both the buffers A and B is, and consequently the lower the ionic strength is, as shown in Table 3, the higher elution pH can be achieved.

TABLE 3
Buffer composition and ionic strengths for Citrate buffers in study:

| | Ionic | | Ionic |
|---|---|---|---|
| | Strength | | Strength |
| Buffer A: | (M) | Buffer B | (M) |
| 50 mM pH 5.5 | 0.1961 | 50 mM pH 2.5 | 0.01046 |
| 20 mM pH 5.5 | 0.0725 | 20 mM pH 2.5 | 0.00404 |
| 10 mM pH 5.5 | 0.03454 | 10 mM pH 2.8 | 0.00341 |

### Example 2-Purification of mAb Using Succinate Buffer and Protein L Separation Matrix

In this example, Protein L chromatography according to the above was performed with the experimental setups 1 and 2 in Table 2. The resulting chromatogram is shown in FIG. 2. It is clear that the lower the concentration of buffer A, and by using the indicated concentration for Buffer B, the lower the ionic strength is, as shown in Table 4, and the higher elution pH can be achieved.

TABLE 4
Buffer compositions and ionic strengths
for Succinate buffers in study:

| | Ionic | | Ionic |
|---|---|---|---|
| | Strength | | Strength |
| Buffer A: | (M) | Buffer B | (M) |
| 10 mM pH 5.8 | 0.0225 | 50 mM pH 2.8 | 0.00198 |
| 20 mM pH 6 | 0.0506 | 50 mM pH 2.8 | 0.00198 |

Here it can be observed in FIG. 2 that the elution pH is significantly increased with about one (1) pH unit compared to the citrate buffers in FIG. 1.

### Example 3-SEC Analysis

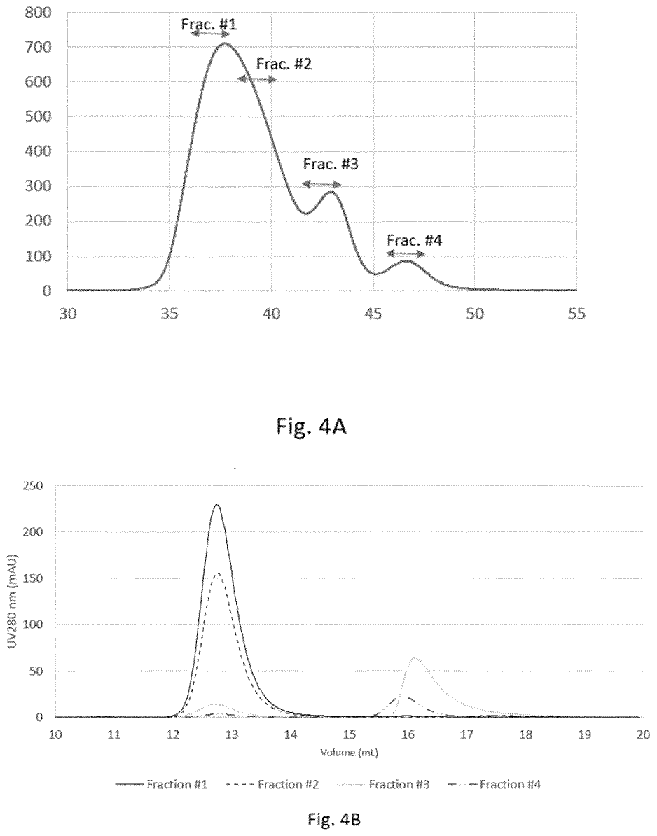

Fractions were collected from the Chromatography experiments above, as indicated in FIG. 4A. The result of the SEC analysis is summarized in Table 5. An exemplary SEC curve, from the 10 mM/50 mM succinate experiment, is shown in FIG. 4B.

TABLE 5
SEC analysis of collected fractions

| Buffer | | HMW | monomeric | LMW |
|---|---|---|---|---|
| (Equilibration/elution) | Fraction # | (%) | mAb (%) | (%) |
| 10 mM/50 mM Succinate | 1 | | 100.0 | 0.0 |
| | 2 | 0.4 | 98.2 | 1.4 |
| | 3 | 1.8 | 16.8 | 81.3 |
| | 4 | | 13.0 | 87.0 |
| 20 mM/50 mm Succinate | 1 | | 99.4 | 0.6 |
| | 2 | 0.4 | 96.3 | 3.3 |
| | 3 | 2.3 | 31.0 | 66.6 |
| | 4 | | 16.3 | 83.7 |
| 10 mM/10 mM Citrate | 1 | | 100.0 | 0.0 |
| | 2 | 1.6 | 49.0 | 49.5 |
| | 3 | | 13.7 | 86.3 |
| 20 mM/20 mM Citrate | 1 | | 95.7 | 4.3 |
| | 2 | 2 | 59.0 | 39.0 |
| | 3 | | 29.7 | 70.3 |
| 50 mM/50 mM Citrate | 1 | | 81.4 | 18.6 |
| | 2 | 1 | 76.3 | 22.6 |
| | 3 | 2 | 23.3 | 74.7 |

It is clear that, for the succinate elution experiments, the first fraction from the main peaks in the chromatograms, with an elution pH of 4.5, comprise the monomeric mAb to be purified at, or close to, 100%. When eluting with citrate buffers, it is clear that the lowest concentration of citrate buffer, 10 mM/10 mM, leads to the highest amount of the monomeric mAb in the first fraction from the main peak (10 mM, elution pH 3.75, 100% target monomer), whereas 50 mM citrate does not show as good a separation of the monomeric mAb from other molecules present in the feed.

In the cases of 10 mM/50 mM succinate, 20 mM/50 mM succinate, the second fraction still comprise a considerable amount of target monomer, but also comprise a small amount of a Low Molecular Weight (LMW) species. Without being bound to any theory, it is believed that this LMW comprise single light chains. Furthermore, the second fraction also comprise a High Molecular Weight (HMW) species. Without being bound to any theory, this is believed to comprise aggregates. The amount of LMW and HMW thereafter increases in the third fractions, as well as fourth fractions.

In the cases of 20 mM/20 mM citrate and 50 mM/50 mM citrate, the first fractions from the main peak also comprise LMW, which increases with the following two fractions.

Again, this shows that low concentration of succinate buffers as specified above, but also the lowest concentrations of citrate buffer, with a low ionic strength as a consequence, leads to a good separation of the monomeric mAb from other species comprised in the feed, such as LMW and HMW.

### Example 4-Separation of Bispecific Antibodies on a Protein L Separation Matrix

### 4A. Comparison of 10 mM Citrate Vs 10 mM Succinate Buffers

In this example, the bsAb as defined above was run with the experimental setups 1 and 5 in Table 2 and on the Protein L separation matrix according to the above. The chromatograms (FIG. 3) show three peaks in the pH gradient. For the 10 mM/50 mM succinate buffer, the three peaks elute at pH 4.8, 4.1 and 3.8, respectively. For the 10 mM/10 mM citrate buffer, the three peaks elute at pH 4.8, 3.9 and 3.6, respectively.

LC-MS analysis (data not shown) shows that the second peak comprises the heterodimeric bispecific antibody comprising one kappa VL chain and one lambda VL chain. The third peak comprised a monomeric Ab comprising two kappa VL chains (homodimer) (data not shown). The first peak comprises species or antibody variants that nonspecifically and/or weakly bind to the separation matrix, including monomeric Ab comprising two lambda VL chains (homodimer).

### 4B. Separation Using 15 mM Succinate Buffer

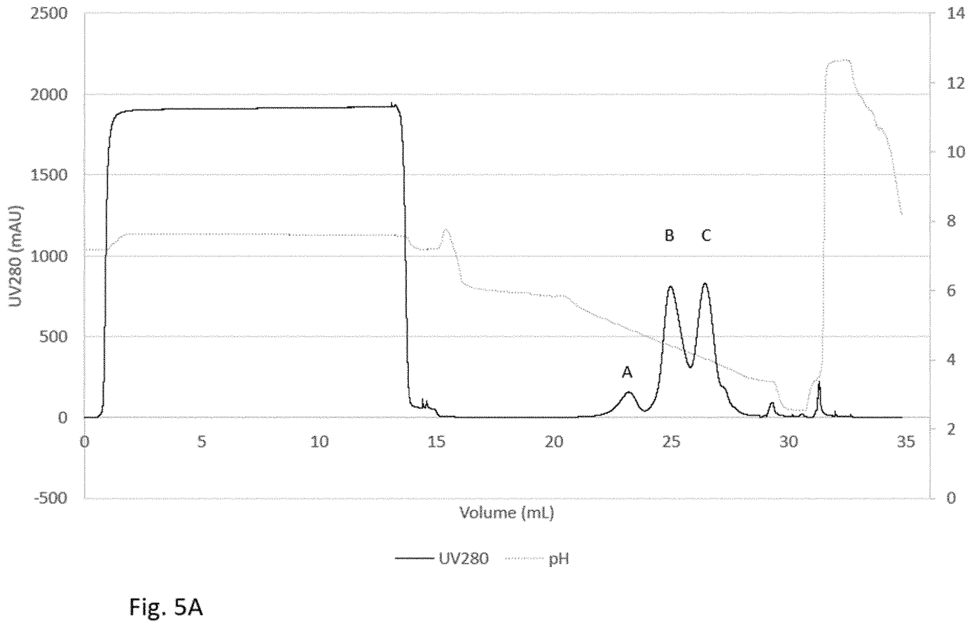

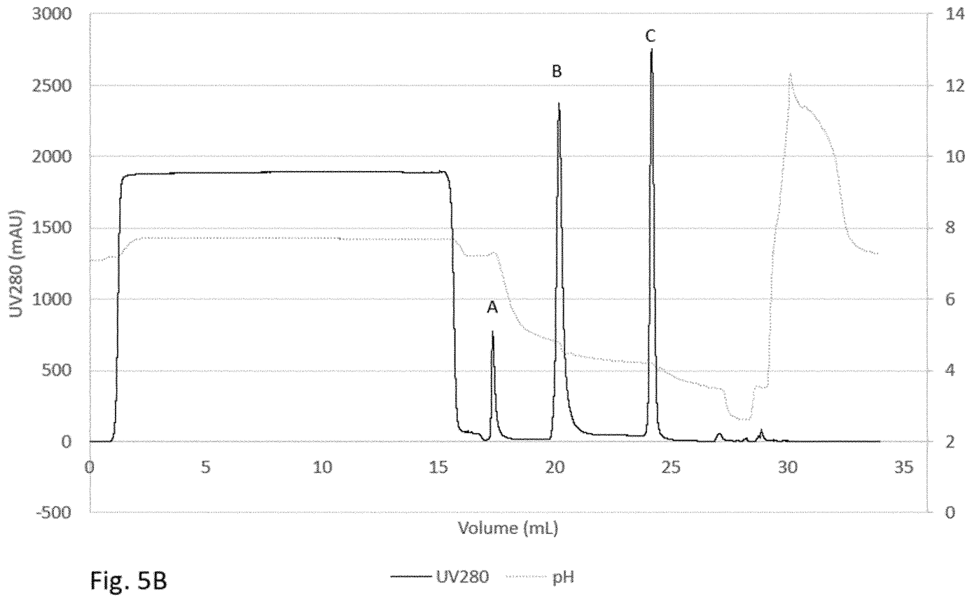

For this example, the bsAb as defined above was run with the experimental setup 7 in Table 2 and on the Protein L separation matrix according to the above. Elution was performed in a gradient (FIG. 5A) of pH 6-3.4, or in a stepwise elution (FIG. 5B). In the stepwise elution, pH 4.8 was used to elute the lambda homodimer, pH 4.3 was used to elute the lambda-kappa heterodimer, and pH 3.4 was used to elute the kappa homodimer.

TABLE 6
Elution pH for the entities in the feed using 15 mM Succinate

| Peak | Entity | Elution pH |
|---|---|---|
| A | Lambda homodimer | 4.9 |
| B | bsAb (heterodimer) | 4.4 |
| C | Kappa homodimer | 4.0 |

As can be seen in FIG. 5A and FIG. 5B, a clear separation is achieved of the heterodimer from the two homodimers.

### 4C. Separation Using 50 mM Propionate Buffer

For this example, the bsAb as defined above was run with the experimental setup 6 in Table 2 and on the Protein L separation matrix according to the above. Elution was performed in a gradient (FIG. 6A) or in a stepwise elution (FIG. 6B). In the stepwise elution, pH 4.8 was used to elute the lambda homodimer, pH 4.4 was used to elute the lambda-kappa heterodimer, and pH 3.5 was used to elute the kappa homodimer.

TABLE 7
Elution pH for the entities in the feed using 50 mM Propionate

| Peak | Entity | Elution pH |
|---|---|---|
| A | Lambda homodimer | 5.0 |
| B | bsAb (heterodimer) | 4.5 |
| C | Kappa homodimer | 4.1 |

As can be seen in FIG. 6A and FIG. 6B, a clear separation is achieved of the heterodimer from the two homodimers.

### Example 5. Purification of mAb: Comparison of 15 mM Succinate, 50 mM Propionate, and 20 mM Citrate

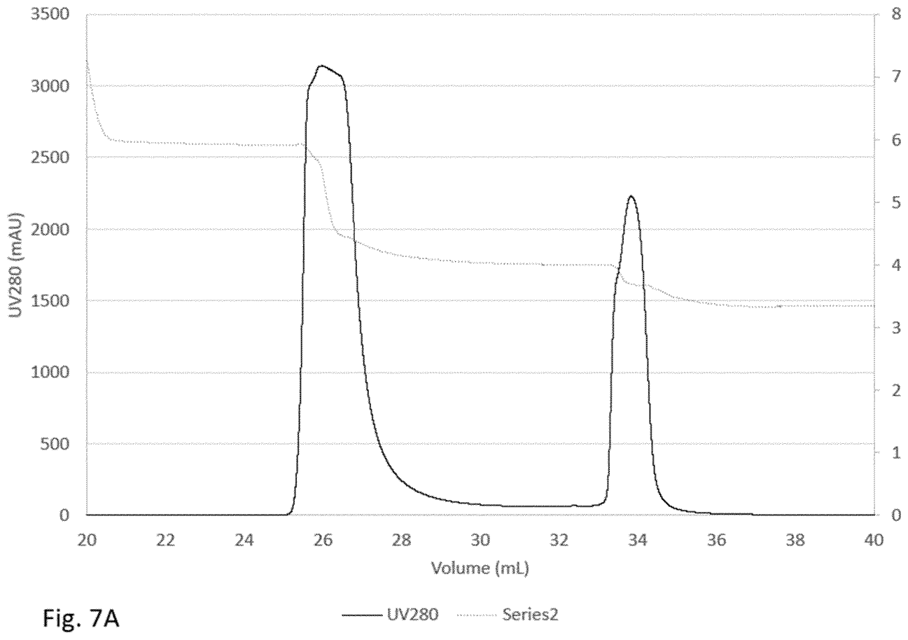

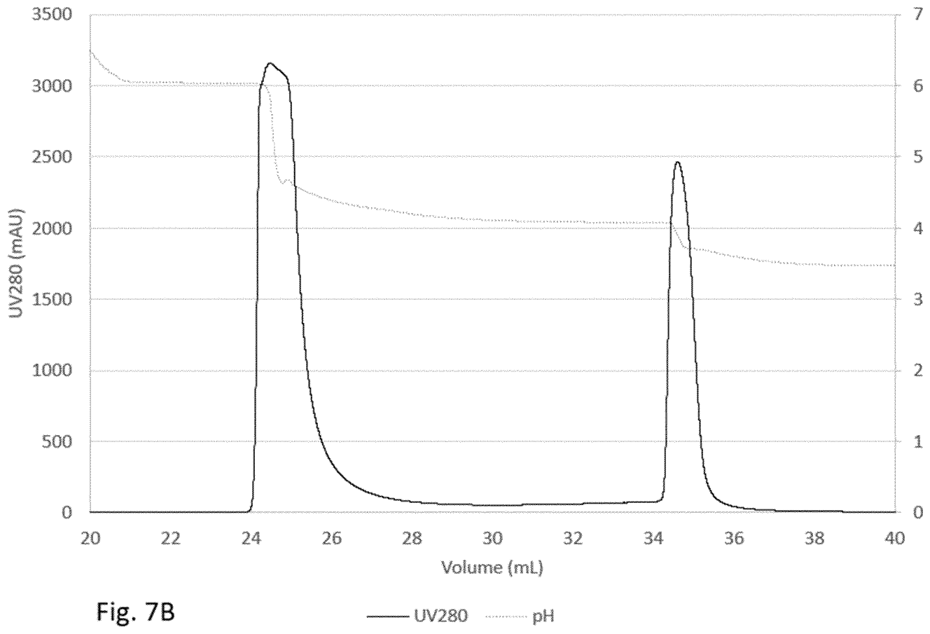

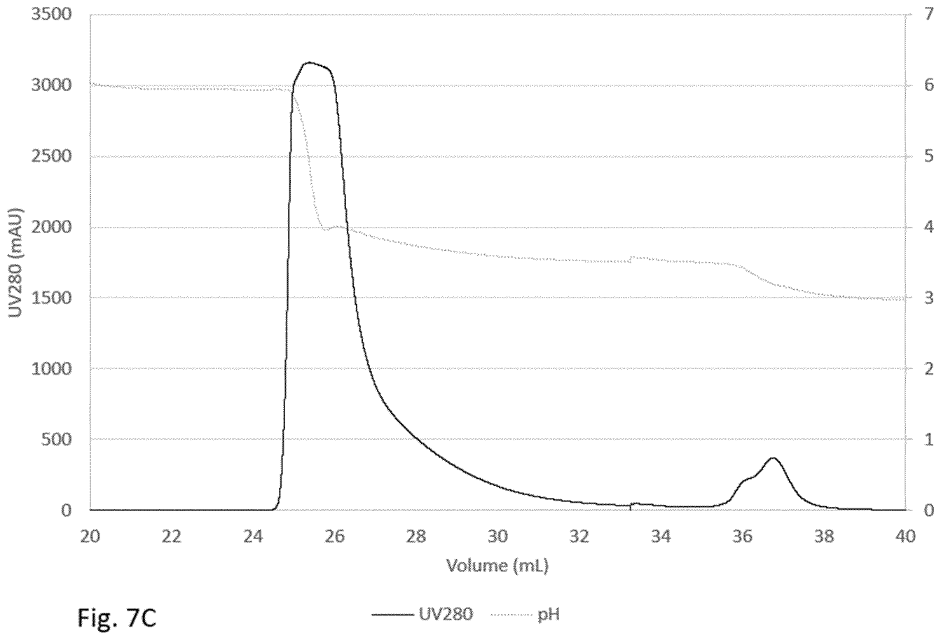

In this example, Protein L chromatography according to the above was performed with the experimental setups 4, 6 and 7 in Table 2. The results of the experiment are summarized in Table 8 below, and the resulting chromatograms are shown in FIG. 7A (15 mM Succinate), FIG. 7B (50 mM Propionate) and FIG. 7C (20 mM Citrate).

TABLE 8

| | | | Pool | Monomer | Monomer |
|---|---|---|---|---|---|
| | | Elution | volume | Yield | purity |
| Peak # | Buffer | pH | (ml) | (%) | (%) |
| 1 | 50 mM Propionate | 4.2 | 3.4 | 87 | 97 |
| (mono- | 15 mM Succinate | 4.1 | 4.1 | 92 | 97 |
| mer) | 20 mM Citrate | 3.6 | 6.3 | 96 | 85 |
| 2 | 50 mM Propionate | 3.5 | 1.4 | 1 | 5.4 |
| (light | 15 mM Succinate | 3.4 | 1.5 | 1 | 5.5 |
| chain) | 20 mM Citrate | 3 | 1.7 | 1 | 23.5 |

Thus, it has been shown that using low concentrations for the elution buffer, lower than commonly used, will increase the elution pH that can be achieved. Additionally, a separation of heterodimeric antibodies from homodimeric antibodies can be achieved, while still achieving the increased elution pH.

Additionally, it has been shown that succinate and propionate buffers are advantageous alternatives to the commonly used citrate buffer. In particular, it has been shown that low concentrations of succinate, and lower concentrations than commonly used for citrate, are advantageous in achieving a higher elution pH on a Protein L separation matrix. Without being bound to any theory, it is believed that the lower ionic strength of the buffer solution is the reason for the higher elution pH. Thus, buffers composed of a mono- or dicarboxylic acid might be advantageous compared to a tricarboxylic acid since the ionic strength at a given pH will be lower for the mono- and dicarboxylic acids. This is due to that less sodium ions (from NaOH titration) are needed to achieve the same pH.

1. A method for purification of an antibody or an antibody fragment, comprising the steps of:
adsorbing at least one of the antibody or the antibody fragment onto an affinity separation matrix by contacting a liquid sample with the affinity separation matrix;
washing the affinity separation matrix to remove impurities;
equilibrating the affinity separation matrix with an equilibration buffer
separating the at least one antibody or antibody fragment from the affinity separation matrix by using an elution buffer,
wherein the affinity separation matrix comprises a ligand based on Protein L, or any variation thereof that binds to a κ-light chain of the antibody or antibody fragment, and wherein the elution buffer has a concentration of 5-50 mM.

2. A method for separation of bispecific antibodies comprising the steps of:
adsorbing a feed comprising at least the bispecific antibody onto an affinity separation matrix by contacting a liquid sample with the affinity separation matrix;
washing the affinity separation matrix to remove impurities;
equilibrating the affinity separation matrix with an equilibration buffer
adding an elution buffer to the separation matrix to elute the bispecific antibody from the affinity separation matrix,
wherein the affinity separation matrix comprises a ligand based on Protein L, or any variation thereof that binds to a κ-light chain of the bispecific antibody, and wherein the elution buffer has a concentration of 5-50 mM.

3. A method for obtaining an elution pH of above 4 from a Protein L or Protein L-derived chromatography separation matrix using an elution buffer based on a carboxylic acid, wherein the elution pH is obtained by lowering the elution buffer concentration to below 50 mM, preferably to between 10-50 mM.

4. The method according to claim 1, wherein the elution buffer comprises a buffering dicarboxylic acid.

5. The method according to claim 4, wherein the elution buffer is a succinate buffer solution.

6. The method according to claim 5, wherein the succinate buffer solution has a concentration of 10-50 mM.

7. The method according to claim 5, wherein the succinate buffer solution has a concentration of 10-20 mM, preferably 15 mM.

8. The method according to claim 5, wherein the pH of the succinate buffer solution is 3-5, or 2.8-5.

9. The method according to claim 1, wherein the elution buffer comprises a buffering tricarboxylic acid.

10. The method according to claim 9, wherein the elution buffer is a citrate buffer solution.

11. The method according to claim 9, wherein the citrate buffer solution has a concentration of 5-45 mM.

12. The method according to claim 9, wherein the pH of the citrate buffer solution is 4.5-2.5.

13. The method according to claim 1, wherein the elution buffer comprises a buffering monocarboxylic acid.

14. The method according to claim 13, wherein the elution buffer is a propionate buffer solution.

15. The method according to claim 14, wherein the propionate buffer solution has a concentration of 50 mM.

16. The method according to claim 1, wherein the elution is performed in a pH range of from about 5 to about 3.
